(19) United States
(12) Patent Application Publication
(10) Pub. No.: US 2026/0209618 A1
(43) Pub. Date: Jul. 23, 2026

(54) METHODS AND SYSTEMS FOR PRODUCING LIGHT OLEFINS AND AROMATICS FROM GAS CONDENSATES

(71) Applicant: SABIC Global Technologies B.V., Bergen OP Zoom (NL)

(72) Inventors: Rajitha VUPPULA, Bangalore (IN); Vidya Sagar GUGGILLA, Bangalore (IN); Ahmed Suliman AL-ZENAIDI, Bangalore (IN); BV VENUGOPAL, Bangalore (IN); Hatem Abdallah BELFADHEL, Bangalore (IN)

(21) Appl. No.: 19/140,652

(22) PCT Filed: Dec. 18, 2023
(86) PCT No.: PCT/IB2023/062884

§ 371 Date: Jun. 18, 2025

(30) Foreign Application Priority Data
Dec. 20, 2022 (EP) 22215175.5

(51) Int. Cl. C10G 69/04; C07C 7/00

(52) U.S. Cl. CPC C10G 69/04; C07C 7/005; C10G 2300/202; C10G 2300/308; C10G 2400/20; C10G 2400/30

(57) ABSTRACT

Embodiments of the present disclosure describe methods and systems of producing light olefins and aromatics from a gas condensate feedstock using minimal pretreatment of the gas condensate feedstock. The gas condensate feedstock is separated into several streams including a heavy cut stream to produce aromatics and a light cut stream to produce lighter olefins. Other streams can be produces to maximize production of aromatics and lighter olefins depending on the method used.

### TECHNICAL FIELD

The present disclosure relates to producing light olefins and aromatics from gas condensates.

### BACKGROUND

The production of gas condensates around the world has steadily increased in the last few decades. Gas condensate is a byproduct of both gas and oil production. This gas condensate is supplied to a refinery to be processed into intermediate products, such as aromatics and olefins. The intermediate products use fluidized catalytic cracking (FCC) processes, where heavy feedstocks, such as gas oils or residues, are converted along with the gas condensates. However, the gas condensate, which contains large amounts of lighter components, is often a limitation on equipment revamps, catalyst composition in the catalytic cracker, and other refinery processes. Using a conventional FCC process, high gasoline yields are produced. As demand rises for intermediate products, production of the intermediate products beyond using traditional FCC processes are needed.

### SUMMARY

Applicant has recognized that intermediate products, such as light olefins and aromatics can be produced in high yields by utilization of a feedstock which is substantially all gas condensates, instead of a blend of the gas condensate with crude oil. The gas condensate typically has an API gravity (American Petroleum Institute gravity) ranging from about 45 to 70 to produce light olefins and aromatics. Methods and system disclosed here accommodate this feedstock containing large amounts of lighter components as compared to a blend of crude oil and gas condensate to produce the light olefins and aromatics.

In certain embodiments, a method for production of light olefins and aromatics from a gas condensate feedstock includes the step of supplying (1) a hydrogen stream and (2) a gas condensate feedstock containing ethane, propane, butane, light naphtha, and nitrogen- and sulfur-containing impurities to a hydrotreating unit to produce a purified feedstock. The hydrotreating unit substantially removes the nitrogen and sulfur containing impurities from the gas condensate feedstock to produce the purified feedstock. The method further includes the step of supplying the purified feedstock to a splitter. The splitter is configured for separating a light cut stream and a heavy cut stream from the purified feedstock. The light cut stream contains a first plurality of hydrocarbons with a boiling point less than about 300° C. and the heavy cut stream contains a second plurality of hydrocarbons with a boiling point greater than 300° C. The method further includes the step of supplying the light cut stream to a catalytic cracking unit to produce a cracked product stream containing ethane, propane, and aromatics. In certain embodiments, a weight percent ratio of a catalyst to the light cut stream in the catalytic cracking unit ranges from 10:1 to 50:1. A catalyst of the catalytic cracking unit may contain an FCC type based catalyst or a USY based catalyst and an FCC additive or a Mesoporous P2O5-HZSM-5 based catalyst. The FCC type based catalyst may contain an amorphous, porous solid acid matrix and a USY based zeolite, and the FCC additive may contain a Zeolite Socony Mobil (ZSM)-5 and a Mesoporous P2O5-HZSM-5 zeolite. In certain embodiments, a catalyst of the catalytic cracking unit can contain a mesoporous P2O5-HZSM-5 based spray dried catalyst. The method also includes the step of supplying a first portion of the cracked product stream to a steam cracker to produce a first olefin product stream containing ethylene, propylene, and aromatics. In certain embodiments, the first olefin product stream contains about two to ten times weight percent more of ethylene and propylene by weight percent as compared to the cracked product stream. A second portion of the cracked product stream is supplied from the steam cracker to a gas concentration unit for further processing to produce a second olefin product stream containing propylene and aromatics. The method can further include an optional step of supplying the heavy cut stream from the splitter to a catalytic reformer unit to produce an aromatics-enriched stream and a light hydrocarbon stream. The catalytic reformer unit can contain a catalytic reformer and a separation apparatus. In certain embodiments, the separation apparatus can be a distillation column. The method can also include an optional step of combining the light hydrocarbon stream with the light cut stream from the splitter before processing in the catalytic cracking unit. In certain embodiments, the method may include supplying the aromatics-enriched stream to an aromatic recovery complex for production of benzene, toluene, and xylene.

In certain embodiments, a method for production of light olefins and aromatics from a gas condensate feedstock includes the step of supplying a hydrogen stream and a gas condensate feedstock containing ethane, propane, butane, light naphtha, and nitrogen- and sulfur-containing impurities to a hydrotreating unit for substantial removal of the nitrogen- and sulfur-containing impurities from the gas condensate feedstock to produce a purified feedstock. The method also includes supplying the purified feedstock to a splitter and separating a light cut stream, a medium cut stream, and a heavy cut stream from the purified feedstock. The light cut stream contains a first plurality of hydrocarbons with a boiling point less than 30° C. The medium cut stream contains a second plurality of hydrocarbons with a boiling point ranging from 30° C. to 300° C. The heavy cut stream contains a third plurality of hydrocarbons with a boiling point greater than 300° C. The method further includes the step of supplying the light cut stream to a steam cracker to produce a first olefin product stream containing ethylene, propylene, and aromatics. The method also includes the step of supplying the medium cut stream to a catalytic cracking unit to produce a cracked product stream containing ethane, propane, and aromatics. The method further includes the step of supplying a first portion of the cracked product stream to the steam cracker to produce the first olefin product stream containing ethylene, propylene, and aromatics. The steam cracker is also configured for supplying a second portion of the cracked product stream to a gas concentration unit for further processing to produce a second olefin product stream containing the propylene and the aromatics. In certain embodiments, the method further includes an optional step of supplying the heavy cut stream from the splitter to a catalytic reformer unit to produce an aromatics-enriched stream and a light hydrocarbon stream. The method also includes an optional step of combining the light hydrocarbon stream with the medium cut stream from the splitter before processing in the catalytic cracking unit. The method further includes an optional step of supplying the aromatics-enriched stream to an aromatic recovery complex for production of benzene, toluene, and xylene. In certain embodiments, the method can further include recycling a first portion of the aromatics-enriched stream to the hydrotreating unit and supplying a second portion of the aromatics-enriched stream to an aromatic recovery complex for production of benzene, toluene, and xylene. In certain embodiments, the method can further include supplying a first portion of the aromatics-enriched stream as a fuel to a regenerator of the catalytic cracking unit and supplying a second portion of the aromatics-enriched stream to an aromatic recovery complex for production of benzene, toluene, and xylene. In certain embodiments, a catalyst of the catalytic cracking unit can contain an FCC type based catalyst or USY based catalyst and an FCC additive or Mesoporous P₂O₅-HZSM-5 based catalyst. The FCC catalyst may contain an amorphous, porous solid acid matrix and a USY based zeolite, and the FCC additive may contain a Zeolite Socony Mobil (ZSM)-5 and Mesoporous P₂O₅-HZSM-5 zeolite. In certain embodiments, a catalyst of the catalytic cracking unit contains a mesoporous P₂O₅-Zeolite Socony Mobil-5-based spray dried catalyst. In certain embodiments, a weight percent ratio of catalyst to the light cut stream in the catalytic cracking unit may range from 10:1 to 50:1. The first olefin product stream may contain about two to ten times more of ethylene and propylene by weight percent as compared to ethylene and propylene content in the cracked product stream. The first olefin product stream may contain about one to five times more of ethylene by weight percent as compared to ethylene content in the cracked product stream. The first olefin product stream may contain about 50 wt. % to about 80 wt. % more propylene as compared to propylene content in the cracked product stream.

Certain embodiments of the methods include recovering a remaining heavy feed stream from the catalytic reformer unit and recycling the remaining heavy feed stream to the hydrotreating unit. Certain embodiments of the methods include recovering a remaining heavy feed stream from the catalytic reformer unit and supplying the remaining heavy feed stream as a fuel to a regenerator of the catalytic cracking unit. The catalytic reformer unit can contain a catalytic reformer and a separation apparatus. In certain embodiments, the separation apparatus can be a distillation column.

Certain embodiments include olefin and aromatics production systems using a gas condensate feedstock. One such system includes a hydrotreating unit configured for receiving a hydrogen stream and a gas condensate feedstock containing ethane, propane, butane, light naphtha, and nitrogen- and sulfur-containing impurities. The hydrotreating unit removes the nitrogen- and sulfur-containing impurities from the gas condensate feedstock and produces a purified feedstock. The system also includes a splitter in fluid communication with the hydrotreating unit and configured for receiving the purified feedstock and separating the purified feedstock into a light cut stream and a heavy cut stream. The light cut stream contains a first plurality of hydrocarbons with a boiling point less than about 300° C. The heavy cut stream contains a second plurality of hydrocarbons with a boiling point greater than 300° C. The system further includes a catalytic cracking unit in fluid communication with the splitter and configured for receiving the light cut stream and producing a cracked product stream containing a portion of the ethane, the propane, and the aromatics. The system also includes a steam cracker in fluid communication with the catalytic cracking unit and configured for receiving a first portion of the cracked product stream and producing a first olefin product stream containing ethylene, propylene, and aromatics. The system further includes a gas concentration unit in fluid communication with the catalytic cracking unit and configured for receiving a second portion of the cracked product stream and producing a second olefin product stream containing the propylene and the aromatics. The system can also optionally include a catalytic reformer unit in fluid communication with the splitter and configured for receiving the heavy cut stream and producing an aromatics-enriched stream and a light hydrocarbon stream. The catalytic reformer unit can contain a catalytic reformer and a separation apparatus. In certain embodiments, the separation apparatus can be a distillation column. The light hydrocarbon stream combines with the light cut stream from the splitter prior to the catalytic cracking unit. The system can also optionally include an aromatic recovery complex in fluid communication with catalytic reformer unit and configured for receiving the aromatics-enriched stream and producing benzene, toluene, and xylene.

Another embodiment of the olefin and aromatics production system using a gas condensate feedstock includes a hydrotreating unit configured for receiving a hydrogen stream and a gas condensate feedstock containing ethane, propane, butane, light naphtha, and nitrogen and sulfur containing impurities. The hydrotreating unit removes the nitrogen- and sulfur-containing impurities from the gas condensate feedstock and produces a purified feedstock. The system further includes a splitter in fluid communication with the hydrotreating unit and configured for receiving the purified feedstock and separating the purified feedstock into (i) a light cut stream, (ii) a medium cut stream, and (iii) a heavy cut stream. The light cut stream contains a first plurality of hydrocarbons with a boiling point less than about 30° C. The medium cut stream contains a second plurality of hydrocarbons with a boiling point ranging from 30° C. to 300° C. The heavy cut stream contains a third plurality of hydrocarbons with a boiling point greater than 300° C. The system also includes a catalytic cracking unit in fluid communication with the splitter and configured for receiving the medium cut stream and producing a cracked product stream containing a portion of the ethane, the propane, and the aromatics. The system further includes a steam cracker in fluid communication with the splitter and the catalytic cracking unit and configured for receiving the light cut stream and a first portion of the cracked product stream, respectively, and producing a first olefin product stream containing ethylene, propylene, and aromatics. The system also includes a gas concentration unit in fluid communication with the catalytic cracking unit and configured for receiving a second portion of the cracked product stream and producing a second olefin product stream containing propylene and aromatics. In certain embodiments, the system may further include a catalytic reformer unit in fluid communication with the splitter and configured for receiving the heavy cut stream and producing an aromatics-enriched stream and a light hydrocarbon stream. The light hydrocarbon stream may be combined with the medium cut stream from the splitter prior to the catalytic cracking unit. In certain embodiments, the system may also include an aromatic recovery complex in fluid communication with the catalytic reformer unit and configured for receiving the aromatics-enriched stream and producing benzene, toluene, and xylene. The catalytic reformer unit can contain a catalytic reformer and a separation apparatus. In certain embodiments, the separation apparatus can be a distillation column.

Certain embodiments of the systems can include the optional catalytic reformer unit configured for producing an aromatics-enriched stream, a light hydrocarbon stream, and a remaining heavy feed stream. The remaining heavy feed stream may be recycled to the hydrotreating unit or supplied to a regenerator of the catalytic cracking unit as fuel.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is an illustrative diagram of a light olefins and aromatics production system using gas condensate feedstock that is split into two streams including a light cut stream and a heavy cut stream, according to an embodiment of the disclosure.

FIG. 2 is an illustrative diagram of a light olefins and aromatics production system using gas condensate feedstock that is split into three streams including a light cut stream, a medium cut stream, and a heavy cut stream, according to an embodiment of the disclosure.

FIG. 3 is a diagrammatic representation of a method of producing light olefins and aromatics production system using gas condensate feedstock that is split into two streams including a light cut stream and a heavy cut stream, according to an embodiment of the disclosure.

FIG. 4 is a diagrammatic representation of a method of producing light olefins and aromatics production system using gas condensate feedstock that is split into three streams including a light cut stream, a medium cut stream, and a heavy cut stream, according to an embodiment of the disclosure.

### DETAILED DESCRIPTION

The present disclosure provides for methods and systems for producing light olefins and aromatics. So that the manner in which the features and advantages of the embodiments of the methods and systems disclosed herein, as well as others, which will become apparent, may be understood in more detail, a more particular description of embodiments of methods and systems is provided. In the following description, numerous details are set forth in order to provide a thorough understanding of the various embodiments. In other instances, well-known processes, devices, and systems may not been described in particular detail in order not to unnecessarily obscure the various embodiments. Additionally, illustrations of the various embodiments may omit certain features or details in order to not obscure the various embodiments.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.” The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “about” is defined as being close to as understood by one of ordinary skill in the art. In one non-limiting embodiment the terms are defined to be within 10%, preferably, within 5%, more preferably, within 1%, and most preferably, within 0.5%. The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component. The term “substantially” and its variations are defined to include ranges within 10%, within 5%, within 1%, or within 0.5%.

The present disclosure is generally directed to embodiments of methods and systems for producing light olefins and aromatics from gas condensates. Most of the Middle East region based gas condensate feedstock are equivalent to light and sweet crude (API 45-70). Following feed pretreatment, the gas condensate feedstock can be sent to splitter units to separate into different cuts including a light cut stream and a heavy cut stream. The heavy cut stream having a temperature greater than 300° C., can have a high aromatic content that produces aromatics using a catalytic reformer unit. The light cut stream can have both high n-paraffinic and high iso-paraffinic content that is suitable for the catalytic cracking reactor. Existing FCC configurations blend these gas condensate feedstocks with crude oil due to limitations in the operating conditions. The embodiments disclosed herein can accommodate the gas condensate feedstock alone in a catalytic cracker. The gas condensate contains large amounts of components which includes n-paraffin's and iso-paraffins. Lighter feedstock i.e., ethane and propane may be processed in the FCC unit downstream to a steam cracker.

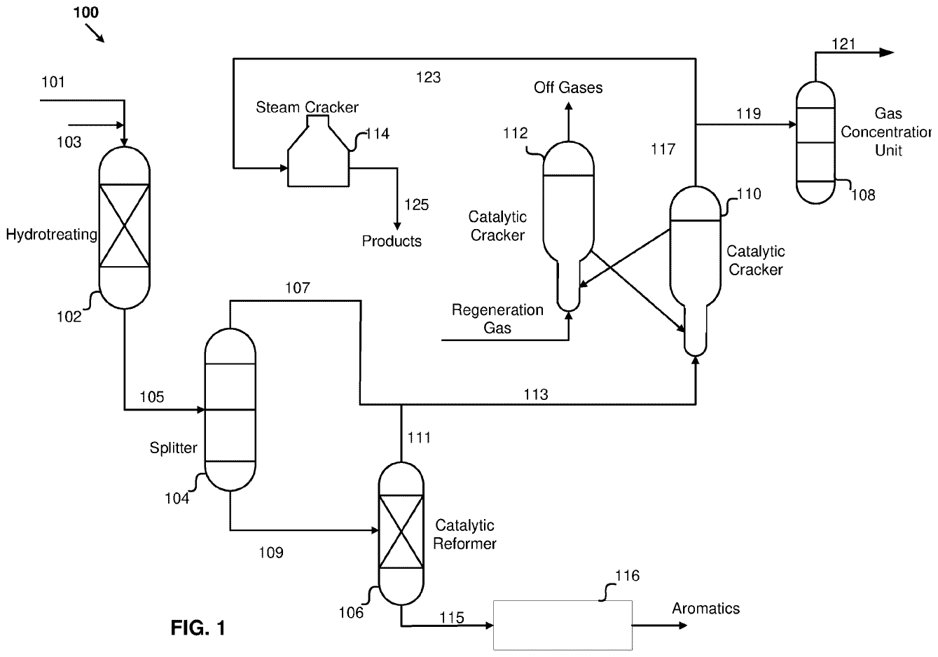

FIG. 1 is an illustrative diagram of a light olefins and aromatics production system 100 using a gas condensate feedstock that is split into two streams including a light cut stream and a heavy cut stream. In certain embodiments, an olefin and aromatics production system using a gas condensate feedstock contains a hydrotreating unit 102. The hydrotreating unit 102 is configured for receiving a hydrogen stream 101 and a gas condensate feedstock 103. The gas condensate feedstock 103 contains ethane, propane, butane, light naphtha, and nitrogen and sulfur containing impurities, which constitute a low-density mixture of hydrocarbon liquids present as gaseous components. In certain embodiments, the gas condensate feedstock contains about 10 weight percent (wt. %) to about 90 wt. % of ethane, propane, butane, and light naphtha. In general, gas condensate feedstock also contains hydrocarbons ranging from C4 to C15. In certain embodiments, the gas condensate contains about 10 wt. % to about 50 wt. % of C6-C15 organic compounds. The gas condensate may contain about 5 wt. % to 40 wt. % aromatics. Aromatics may be monoaromatics or diaromatics. However, the gas condensate feedstock can vary depending on the wet gas field, in which the gas condensate feedstock is applied in system 100. The hydrotreating unit 102 is also configured removing the nitrogen- and sulfur-containing impurities, which may be present in the gas condensate feedstock and producing a purified feedstock 105. It is generally desirable to remove the nitrogen- and sulfur-containing impurities prior to catalytic cracking, as these impurities can poison the catalyst and affect the downstream systems. A catalyst of the catalytic cracking unit may contain an FCC type based catalyst or a USY based catalyst and an FCC additive or a Mesoporous P2O5-HZSM-5 based catalyst. The FCC type based catalyst may contain an amorphous, porous solid acid matrix and a USY based zeolite, and the FCC additive may contain a Zeolite Socony Mobil (ZSM)-5 and a Mesoporous P2O5-HZSM-5 zeolite. In certain embodiments, a catalyst of the catalytic cracking unit can contain a mesoporous P2O5-HZSM-5 based spray dried catalyst.

A splitter 104 is in fluid communication with the hydrotreating unit 102. In this embodiment, the splitter 104 is configured for receiving the purified feedstock 105 and separating the purified feedstock 105 into a light cut stream 107 and a heavy cut stream 109. Separation of the light cut stream 107 and the heavy cut stream 109 is favored in certain embodiments due to the catalytic reformer unit's ability to process the heavy cut stream to produce the aromatic products, such as benzene, toluene, and xylene after further processing. The light cut stream 107 contains a first plurality of hydrocarbons with a boiling point less than about 300° C. The heavy cut stream 109 contains a second plurality of hydrocarbons with a boiling point greater than 300° C. The system 100 further contains a catalytic cracking unit 110 and 112 that is in fluid communication with the splitter 104. The catalytic cracking unit has two components the reactor 110 and the regenerator 112. The catalytic cracking unit 110 and 112 is configured for receiving the light cut stream 107 and producing a cracked product stream 117 that contains ethane, propane, and aromatics. In some embodiments, the light cut stream 107 enters a heat exchanger before feeding to the catalytic cracking unit 110 and 112.

The system also contains a steam cracker 114 that is in fluid communication with the catalytic cracking unit 110 and 112. The steam cracker 114 is configured for receiving a first portion of the cracked product stream 123 and producing a first olefin product stream 125 that contains ethylene, propylene, and aromatics.

The system 100 also contains a gas concentration unit 108 that is in fluid communication with the catalytic cracking unit 110 and 112. The gas concentration unit 108 is configured for receiving a second portion of the cracked product stream 119. The gas concentration unit 108 is also configured for producing a second olefin product stream 121 that contains propylene and aromatics.

In system 100, a catalytic reformer unit 106 is in fluid communication with the splitter 104. The catalytic reformer unit 106 is configured for receiving the heavy cut stream 109 and producing an aromatics-enriched stream 115 and a light hydrocarbon stream 111. The catalytic reformer unit can contain a catalytic reformer and a separation apparatus. In certain embodiments, the separation apparatus can be a distillation column. The light hydrocarbon stream 111 from the catalytic reformer unit 106 combines with the light cut stream 107 from the splitter 104 prior to the catalytic cracking unit 110 and 112. In some embodiments, the combined stream including the light hydrocarbon stream 111 and the light cut stream 107 enters a heat exchanger before feeding to the catalytic cracking unit 110 and 112. The light cut stream 107 may be preheated in a heat exchanger to a temperature ranging from about 300° C. to about 800° C. or the light cut stream 107 may be send to a convection section of the steam cracker unit. Heating of the light cut stream 107 assists with achieving the required feed temperature for the steam cracker. The system 100 further contains an aromatic recovery complex 116 in fluid communication with catalytic reformer unit 106. The aromatic recovery complex 116 is configured for receiving the aromatics-enriched stream 115 and producing benzene, toluene, and xylene. In certain embodiments, after extraction of aromatics in the catalytic reformer unit 106, a remaining heavy feed may be recovered to be used as a fuel for the catalytic regenerator. In certain embodiments, after extraction of aromatics in the catalytic reformer unit 106, a remaining heavy feed stream may be recycled to the hydrotreater. In certain embodiments, the remaining heavy feed stream may contain unconverted feed.

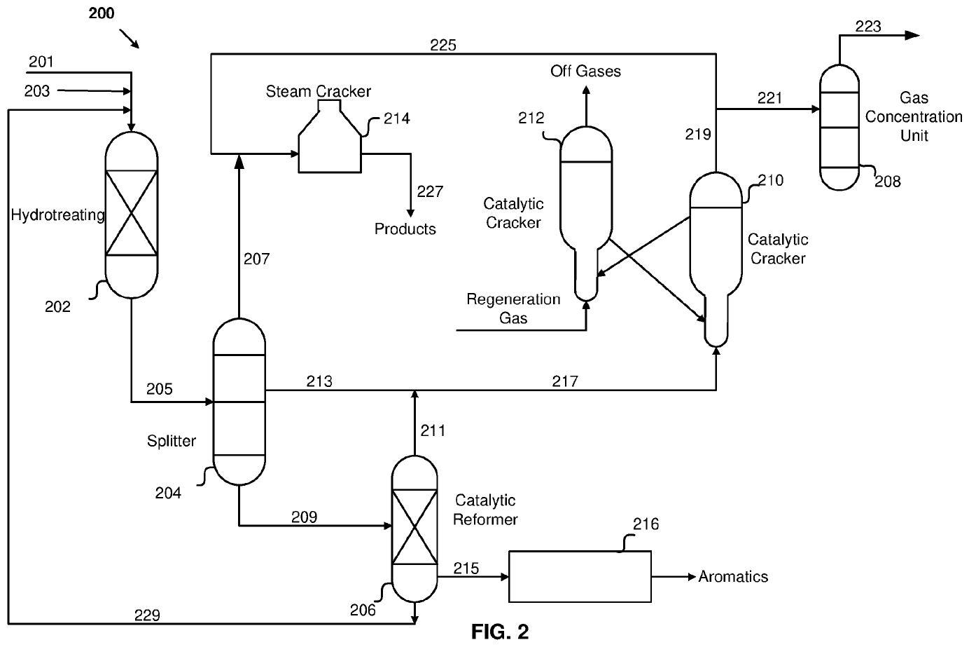

FIG. 2 is an illustrative diagram of a light olefins and aromatics production system 200 using gas condensate feedstock that is split into three streams including a light cut stream, a medium cut stream, and a heavy cut stream. In certain embodiments, an olefin and aromatics production system using a gas condensate feedstock contains a hydrotreating unit 202. The hydrotreating unit 202 is configured for receiving a hydrogen stream 201 and a gas condensate feedstock 203. The gas condensate feedstock 203 contains ethane, propane, butane, light naphtha, and nitrogen and sulfur containing impurities. In general, gas condensate feedstock contains hydrocarbons ranging from C4 to C15. In certain embodiments, the gas condensate contains 25-95 wt. % of C6-C15 organic compounds. However, the gas condensate feedstock can vary depending on the wet gas field, in which the gas condensate feedstock is applied in system 200.

The hydrotreating unit 202 is also configured for removing the nitrogen- and sulfur-containing impurities from the gas condensate feedstock and producing a purified feedstock 205. A splitter 204 is in fluid communication with the hydrotreating unit 202. In the embodiment, the splitter 204 is configured for receiving the purified feedstock 205 and separating the purified feedstock 205 into a light cut stream 207, a middle cut stream 213 and a heavy cut stream 209. In the embodiment, the light cut stream 207 contains a first plurality of hydrocarbons with a boiling point less than about 30° C. The medium cut stream 104C contains a second plurality of hydrocarbons with a boiling point ranging from 30° C. to 300° C. The heavy cut stream 209 contains a third plurality of hydrocarbons with a boiling point greater than 300° C. The system 200 further includes a catalytic cracking unit 210 and 212 that is in fluid communication with the splitter 204. The catalytic cracking unit 210 and 212 is configured for receiving the medium cut stream 213 and producing a cracked product stream 219. The cracked product stream 219 contains ethane, propane, and aromatics. In some embodiments, the light cut stream 207 enters a heat exchanger before feeding to the catalytic cracking unit 210 and 212. The light cut stream 207 may be preheated in a heat exchanger to a temperature ranging from about 300° C. to about 800° C. or the light cut stream 207 may be send to a convection section of the steam cracker unit. Heating of the light cut stream 207 assists with achieving the required feed temperature for the steam cracker.

The system 200 also includes a steam cracker 214 in fluid communication with the splitter 204 and the catalytic cracking unit 210 and 212. The steam cracker 214 is configured for receiving the light cut stream 207 and a first portion of the cracked product stream 225, respectively. The steam cracker 214 is also configured for producing a first olefin product stream 227 containing ethylene, propylene, and aromatics. Further in system 200 is a gas concentration unit 208 in fluid communication with the catalytic cracking unit 210 and 212. The gas concentration unit 208 is configured for receiving a second portion of the cracked product stream 221 and producing a second olefin product stream 223. The second olefin product stream 223 contains propylene and aromatics.

The system 200 can also optionally include a catalytic reformer unit 206 that is in fluid communication with the splitter 204. The catalytic reformer unit 206 is configured for receiving the heavy cut stream 209 and producing an aromatics-enriched stream 215 and a light hydrocarbon stream 211. The catalytic reformer unit can contain a catalytic reformer and a separation apparatus. In certain embodiments, the separation apparatus can be a distillation column. In the embodiment, the light hydrocarbon stream 211 combines with the medium cut stream 213 from the splitter 204 prior to the catalytic cracking unit 210 and 212. In some embodiments, the combined stream including the light hydrocarbon stream 211 and the medium cut stream 213 enters a heat exchanger before being supplied to the catalytic cracking unit 210 and 212. The system 200 can also optionally include an aromatic recovery complex 216 that is in fluid communication with the catalytic reformer unit 206. The aromatic recovery complex 216 is configured for receiving the aromatics-enriched stream 215 and producing benzene, toluene, and xylene. The catalytic reformer unit can contain a catalytic reformer and a separation apparatus. In certain embodiments, the separation apparatus can be a distillation column.

In certain embodiments, after extraction of aromatics in the catalytic reformer unit 206, a remaining heavy feed may be recovered to be used as a fuel for the catalytic regenerator 212. In certain embodiments, after extraction of aromatics in the catalytic reformer unit 206, a remaining heavy feed stream 229 may be recycled to the hydrotreater. In certain embodiments, the remaining heavy feed stream 229 may contain unconverted feed.

Inclusion of a catalytic reformer unit in embodiments of systems 100 or 200 depends on the composition of the gas condensate feedstock. As a non-limiting example, the catalytic reformer unit is included in a system that is processing a gas condensate feedstock containing greater than about 10 wt. % of the heavy cut content. As a non-limiting example, the catalytic reformer unit is included in a system that is processing a gas condensate feedstock containing greater than 10 wt. % of hydrocarbons with a boiling point greater than 300° C.

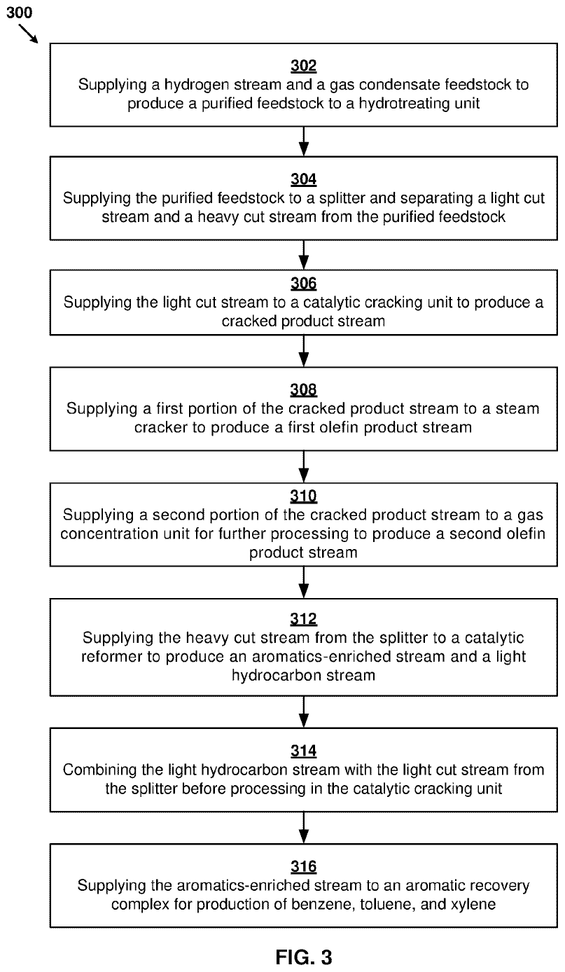

FIG. 3 is a diagrammatic representation of a method 300 for the production of light olefins and aromatics from a gas condensate feedstock that is split into two streams including a light cut stream and a heavy cut stream. This method includes the step 302 of supplying a hydrogen stream and a gas condensate feedstock containing ethane, propane, butane, light naphtha, and nitrogen- and sulfur-containing impurities to a hydrotreating unit. The hydrotreating unit is configured for substantial removal of the nitrogen- and sulfur-containing impurities from the gas condensate feedstock to produce a purified feedstock. In a subsequent step 304, the method includes supplying the purified feedstock to a splitter. The splitter is configured for separating a light cut stream and a heavy cut stream from the purified feedstock. The light cut stream contains a first plurality of hydrocarbons with a boiling point less than about 300° C. The heavy cut stream contains a second plurality of hydrocarbons with a boiling point greater than 300° C. The method 300 further includes the step 306 of supplying the light cut stream to a catalytic cracking unit. The catalytic cracking units produces a cracked product stream that contains ethane, propane, and aromatics. The method also includes the step 308 of supplying a first portion of the cracked product stream to a steam cracker. The steam cracker produces a first olefin product stream containing ethylene, propylene, and aromatics. The method further includes the step 310 of supplying a second portion of the cracked product stream to a gas concentration unit for further processing. The gas concentration unit produces a second olefin product stream containing the propylene and the aromatics.

The method 300 also includes the optional step 312 of supplying the heavy cut stream from the splitter to a catalytic reformer unit. The catalytic reformer unit produces an aromatics-enriched stream, and a light hydrocarbon stream. The catalytic reformer unit can contain a catalytic reformer and a separation apparatus. In certain embodiments, the separation apparatus can be a distillation column. The method further includes the step 314 of combining the light hydrocarbon stream with the light cut stream from the splitter before processing in the catalytic cracking unit. The method further includes the optional step 316 of supplying the aromatics-enriched stream to an aromatic recovery complex for production of benzene, toluene, and xylene.

In some embodiments, the gas condensate feedstock can be substantially all gas condensate with an API gravity ranging from 45 to 70. In other embodiments, a catalyst of the catalytic cracking unit contains an FCC type based catalyst or a USY based catalyst and an FCC additive or a Mesoporous P2O5-HZSM-5 based catalyst. As such, the FCC type based catalyst can contain an amorphous, porous solid acid matrix and a USY based zeolite. The FCC additive can contain a Zeolite Socony Mobil (ZSM)-5 and a Mesoporous P2O5-HZSM-5 zeolite. Mesoporous P2O5-HZSM-5 or ZSM-5 (FCC additive) based spray dried catalyst can crack light gas condensate feedstock i.e., C4-C14 and USY based spray dried (FCC type) catalyst can crack heavier gas condensate feedstock, along with highly branched higher iso-paraffins such as vacuum residue, atmospheric residue, Middle distillate, etc.

In yet another embodiment, where the gas condensate feedstock is substantially all gas condensate, a catalyst of the catalytic cracking unit can contain a mesoporous P₂O₅-HZSM-5 based spray dried catalyst. In these embodiments, a weight percent ratio of catalyst to the light cut stream in the catalytic cracking unit can range from 10:1 to 50:1. The weight percent ratio of catalyst to the light cut stream in the catalytic cracking unit can range from 15:1 to 40:1. The weight percent ratio of catalyst to the light cut stream in the catalytic cracking unit can range from 15:1 to 30:1. The weight percent ratio of catalyst to the light cut stream in the catalytic cracking unit can range from 20:1 to 30:1. In another embodiment, the first olefin product stream can contain about two to ten times weight percent more of ethylene and propylene by weight percent as compared to the cracked product stream.

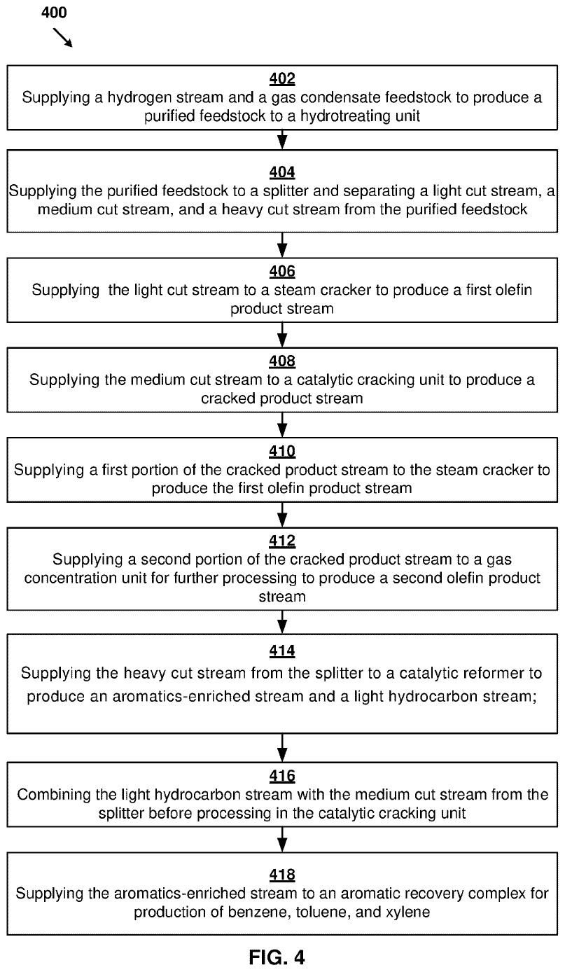

In certain embodiments, when the gas condensate feedstock contains substantial amounts of C1-C4 content, then the gas condensate feedstock may be processed into a light cut, a medium cut, and a heavy cut stream of FIG. 2 or FIG. 4. In certain embodiments, when the gas condensate feedstock does not contain substantial amounts of C1-C4 content, the gas condensate feedstock may be processed into a light cut and a heavy cut stream of FIG. 1 or FIG. 3. There is an increase in the olefins yield when the gas condensate feedstock contains substantial amounts of C1-C4 content. There is an increase in the aromatics yield increase when the gas condensate feedstock does not contain substantial amounts of C1-C4 content.

FIG. 4 is a diagrammatic representation of a method of producing light olefins and aromatics production system using gas condensate that is split into three streams including a light cut stream, a medium cut stream, and a heavy cut stream. The method 400 includes the step 402 of supplying a hydrogen stream and a gas condensate feedstock containing ethane, propane, butane, light naphtha, and nitrogen- and sulfur-containing impurities to a hydrotreating unit. The hydrotreating unit is configured for substantial removal of the nitrogen and sulfur containing impurities from the gas condensate feedstock. The hydrotreating unit produces a purified feedstock. The method further includes the step 404 of supplying the purified feedstock to a splitter. The splitter is configured for separating a light cut stream, a medium cut stream, and a heavy cut stream from the purified feedstock. In the embodiment, the light cut stream contains a first plurality of hydrocarbons with a boiling point less than 30° C., the medium cut stream contains a second plurality of hydrocarbons with a boiling point ranging from 30° C. to 300° C., and the heavy cut stream containing a third plurality of hydrocarbons with a boiling point greater than 300° C.

The method 400 further includes the step 406 of supplying the light cut stream to a steam cracker. The steam cracker produces a first olefin product stream containing ethylene, propylene, and aromatics. The method 400 also includes the step 408 of supplying the medium cut stream to a catalytic cracking unit. The catalytic cracking unit produces a cracked product stream containing ethane, propane, and aromatics. The cracked product stream is then split into two streams, a first portion of the cracked product stream and a second portion of the crack product stream. The method 400 further includes the step 410 of supplying the first portion of the cracked product stream to the steam cracker to produce first olefin product stream containing ethylene, propylene, and aromatics. The method 400 further includes the step 412 of supplying the second portion of the cracked product stream to a gas concentration unit for further processing. The gas concentration unit produces a second olefin product stream containing the propylene and the aromatics.

The method 400 also includes the step 414 of supplying the heavy cut stream from the splitter to a catalytic reformer unit. The catalytic reformer unit produces two streams, an aromatics-enriched stream and a light hydrocarbon stream. The catalytic reformer unit can contain a catalytic reformer and a separation apparatus. In certain embodiments, the separation apparatus can be a distillation column. The method further includes the step 416 of combining the light hydrocarbon stream with the medium cut stream from the splitter before processing in the catalytic cracking unit. The method 400 also includes the step 418 of supplying the aromatics-enriched stream to an aromatic recovery complex for the production of benzene, toluene, and xylene.

In some embodiments, method 400 can further include recycling a first portion of the aromatics-enriched stream to the hydrotreating unit. The first portion of the aromatics-enriched stream can combine with the hydrogen stream and gas condensate stream to produce a purified feedstock. The purified feedstock further can be processed as described herein.

The embodiment can also include supplying a second portion of the aromatics-enriched stream to an aromatic recovery complex for production of benzene, toluene, and xylene. In another embodiment, method 400 can further include supplying a first portion of the aromatics-enriched stream as a fuel to a regenerator of the catalytic cracking unit. The embodiment can also include supplying a second portion of the aromatics-enriched stream to an aromatic recovery complex for production of benzene, toluene, and xylene. In these embodiments, a catalyst of the catalytic cracking unit can contain an FCC type based catalyst or a USY based catalyst and an FCC additive or a Mesoporous P2O5-HZSM-5 based catalyst. The FCC type based catalyst is an amorphous, porous solid acid matrix and a USY based zeolite. The FCC additive is a Zeolite Socony Mobil (ZSM)-5 and a Mesoporous P2O5-HZSM-5 zeolite. These embodiments can also have a catalyst of the catalytic cracking unit that contains a Mesoporous P₂O₅-Zeolite Socony Mobil-5-based spray dried catalyst. In certain embodiments, a weight percent ratio of catalyst to the light cut stream in the catalytic cracking unit can range from 10:1 to 50:1. The weight percent ratio of catalyst to the light cut stream in the catalytic cracking unit can range from 15:1 to 40:1. The weight percent ratio of catalyst to the light cut stream in the catalytic cracking unit can range from 15:1 to 30:1. The weight percent ratio of catalyst to the light cut stream in the catalytic cracking unit can range from 20:1 to 30:1.

In other embodiments, the first olefin product stream can contain about two to ten times more of ethylene and propylene by weight percent as compared to ethylene and propylene content in the cracked product stream. In yet another embodiment, the first olefin product stream can contain about one to five times more of ethylene by weight percent as compared to ethylene content in the cracked product stream. In another embodiment, the first olefin product stream contains about 50 wt. % to about 80 wt. % more of propylene as compared to propylene content in the cracked product stream. Catalytic cracking produces a predominant amount of propylene due to the carbonium ion mechanism. Steam cracking produces a predominant amount of ethylene due to the free radical mechanism.

Embodiments of the systems and methods disclosed herein include systems and methods that do not include a catalytic reformer unit for processing the heavy cut stream before being supplied to a fluid catalytic system. As a non-limiting example, the catalytic reformer unit is included in a system that is processing a gas condensate feedstock containing greater than about 10 wt. % of the heavy cut content. As a non-limiting example, the catalytic reformer unit is included in a system that is processing a gas condensate feedstock containing greater than 10 wt. % of hydrocarbons with a boiling point greater than 300° C. In this example, as the feed has a high heavy content, sending the high cut stream to a catalytic cracker may increase the volumetric processing load for the reactor and cause less than optimal processing. In another non-limiting example, the catalytic reformer unit is not included in a system that is processing a gas condensate feedstock containing less than 10 wt. % of the heavy cut content. In this example, the heavy cut stream from the splitter is supplied to a catalytic cracking unit followed by processing in a steam cracking unit. Embodiments including a catalytic reformer unit are used to improve the aromatics yield when the gas condensate feedstock contains greater than about 10 wt. % of the heavy cut content. Also, processing of the gas condensate feedstock through a catalytic reformer unit may lead to a higher quality feed to the catalytic cracking unit. In certain embodiments, the heavy cut stream from the splitter is recycled to the hydrotreater.

### EXAMPLES

Various examples are described to illustrate selected aspects of the various embodiments of systems and methods for the production of light olefins and aromatics from a gas condensate feed stock. The following examples are intended to be purely exemplary and are not intended to limit the disclosure. There are numerous variations and combinations of reaction conditions, for example, component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

In Example 1, gas condensate feedstock is catalytically cracked into light olefins and aromatics. Typical feedstock properties details are shown below in Table 1.

TABLE 1
Typical gas condensate feedstock properties.

| API | 55.80 | |
|---|---|---|
| Density 15° C. | 0.75 | g/ml |
| Specific Gravity | 0.76 | |
| Refractive Index | 1.42 | |
| Total nitrogen | 9.60 | ppm wt. |
| Sulphur | 198.30 | pm wt. |

Paraffins, Isoparaffins, Olefins, Naphthenes, and Aromatics (PIONA) details are shown below in Table 2.

TABLE 2
PIONA Details.

| PIONA Details | Wt. % |
|---|---|
| Paraffins | 31.11 |
| Iso-paraffins | 31.73 |
| Olefins | 7.78 |
| Naphthenes | 17.06 |
| Aromatics | 12.33 |

The catalytic cracker has an internal diameter of 6.7 millimeters (mm) and a height of 9 meters (m). The catalytic cracker uses mesoporous P2O5-HZSM-5 based spray dried catalyst. At 675° C. with a steam/oil ratio of 0.25 catalyst and a catalyst to feed weight ratio of 25, the product yields are checked. The single pass yields of the cracked product stream from the catalytic cracker are shown below in Table 3 in weight percent (Wt. %).

TABLE 3
Conversion and product yields distribution
with gas condensate in catalytic cracker.

| Products | Wt. % |
|---|---|
| Gasoline (C5 at 421° F.) | 40.01 |
| Coke yield | 1.39 |
| Light cycle oil | 4.7 |
| Hydrogen | 0.36 |
| Methane | 5.85 |
| Ethane | 3.67 |
| Ethylene | 12.76 |
| Propane | 2.67 |
| Propylene | 17.26 |
| Isobutane | 0.66 |
| n-Butane | 1.59 |
| Isobutane | 3.13 |
| n-butanes | 5.13 |
| 1,3 Butadiene | 0.07 |

### Example 2

In Example 2, estimated yields of aromatics, ethylene, and propylene are calculated based on the single pass yields data from Example 1 in Table 3. Ethane and propane is recycled to the steam cracker. The final yields of olefins and aromatics are shown below in Table 4.

TABLE 4
Ultimate yields of olefins and aromatics.

| Products | Wt. % |
|---|---|
| Ethylene | 23.02 |
| Propylene | 26.17 |
| Aromatic | 28.66 |

### Example 3

In Example 3, a composition comparison is shown to demonstrate composition differences between cracking gas condensate using a traditional approach compared to cracking gas condensate into multiple streams, such as a light cut stream and a heavy stream. In the example, two types of Khuff Gas Condensate (KGC) feed composition PIONA details for type-I and type-II are shown in Table 5 and Table 6, respectively in Wt. %.

TABLE 5
Typical PIONA distribution of KGC (Wt. %) type-I.

| | n-Paraffins | Iso-paraffins | Olefins | Naphthenes | Aromatics |
|---|---|---|---|---|---|
| C4 | 3.11 | 0.79 | 0.05 | — | — |
| C5 | 5.23 | 4.25 | — | 0.26 | — |
| C6 | 5.34 | 6.23 | — | 3.47 | 0.76 |
| C7 | 4.86 | 5.30 | 0.10 | 7.69 | 1.52 |
| C8 | 3.96 | 6.18 | 2.84 | 3.29 | 2.98 |
| C9 | 2.86 | 3.26 | 3.52 | 1.49 | 3.30 |
| C10 | 2.18 | 4.31 | 1.00 | 0.86 | 2.37 |
| C11 | 2.02 | 0.90 | 0.27 | — | 1.10 |
| C12 | 1.47 | 0.51 | — | — | 0.30 |

TABLE 6
Typical PIONA Distribution of KGC (Wt. %) type-II.

| | n-Paraffins | Iso-paraffins | Naphthenes | Aromatics |
|---|---|---|---|---|
| C4 | 2.4 | 0.5 | — | — |
| C5 | 4.5 | 3.4 | 0.4 | — |
| C6 | 4.8 | 4.8 | 3.0 | 0.8 |
| C7 | 4.5 | 4.6 | 6.7 | 2.0 |
| C8 | 3.5 | 5.0 | 3.1 | 4.2 |
| C9 | 3.0 | 4.0 | 1.8 | 4.9 |
| C10 | 2.5 | 2.9 | 0.3 | 2.7 |
| C11 | 2.2 | 1.5 | 0.3 | 2.9 |
| C12 | 2.0 | 0.7 | — | 1.9 |
| C13 | 1.6 | 1.5 | — | — |
| C14 | 1.4 | 1.2 | — | — |

Using the data in Table 5, the KGC type-I gas condensate feedstock is cracked using a traditional approach in a catalytic cracker. The catalytic cracker has a reaction temperature of 670° C., a catalyst/feed Wt ratio=30:1, and uses a ZSM-5 catalyst. The composition in Wt. % is shown below in Table 7.

TABLE 7
Single pass yield composition using
KGC type-I gas condensate feedstock.

| Products | Single pass yields (wt %) |
|---|---|
| Ethylene | 12.76 |
| Propylene | 17.26 |
| Coke yield | 1.39 |
| CH4 | 5.85 |
| C6-C8 aromatics | 14.7 |
| Mixed C4's | 11.78 |
| Fraction (36-219° C. w/o C6-C8) aromatics) | 25.30 |

Using KGC-type I gas condensate feedstock in Table 5, the feedstock is cracked using system 100, where the gas condensate is split into a light cut stream and a heavy cut stream. The heavy cut stream, however, is not processed in the catalytic reform unit. As such, additional aromatics can be obtained based on KGC feed composition. The ultimate yield is shown below in Table 8. This Example reports single pass yields from a catalytic cracker followed by ultimate yields using a catalytic cracker & a steam cracker integrated configuration.

TABLE 8
Ultimate yields of KGC type-I feed
using an embodiment of system 100.

| Products | Ultimate yields (wt %) |
|---|---|
| Ethylene | 24.02 |
| Propylene | 26.13 |
| Coke yield | 2.05 |
| CH4 | 9.89 |
| C6-C8 aromatics | 22.01 |

Embodiments of a method for production of light olefins and aromatics from a gas condensate feedstock includes the steps of supplying a hydrogen stream and a gas condensate feedstock containing ethane, propane, butane, light naphtha, and nitrogen and sulfur containing impurities to a hydrotreating unit for substantial removal of the nitrogen and sulfur containing impurities from the gas condensate feedstock to produce a purified feedstock. In certain embodiments, the gas condensate feedstock is substantially all gas condensate with an API gravity ranging from 50 to 70. The method further includes supplying the purified feedstock to a splitter and separating a light cut stream and a heavy cut stream from the purified feedstock. The light cut stream contains a first plurality of hydrocarbons with a boiling point less than about 300° C. and the heavy cut stream containing a second plurality of hydrocarbons with a boiling point greater than 300° C. The method further includes supplying the light cut stream to a catalytic cracking unit to produce a cracked product stream containing ethane, propane, and aromatics; and supplying a first portion of the cracked product stream to a steam cracker to produce a first olefin product stream containing ethylene, propylene, and aromatics, and supplying a second portion of the cracked product stream to a gas concentration unit for further processing to produce a second olefin product stream containing the propylene and the aromatics. In certain embodiments, the first olefin product stream contains about two to ten times weight percent more of ethylene and propylene by weight percent as compared to the cracked product stream. The method may further include an optional step of supplying the heavy cut stream from the splitter to a catalytic reformer unit to produce an aromatics-enriched stream and a light hydrocarbon stream; combining the light hydrocarbon stream with the light cut stream from the splitter before processing in the catalytic cracking unit; and supplying the aromatics-enriched stream to an aromatic recovery complex for production of benzene, toluene, and xylene. The catalytic reformer unit can contain a catalytic reformer and a separation apparatus. In certain embodiments, the separation apparatus can be a distillation column. In certain embodiments, a catalyst of the catalytic cracking unit contains an FCC type based catalyst or a USY based catalyst and an FCC additive or a Mesoporous P2O5-HZSM-5 based catalyst. The FCC type based catalyst may contain an amorphous, porous solid acid matrix and a USY based zeolite, and the FCC additive may contain a Zeolite Socony Mobil (ZSM)-5 and a Mesoporous P2O5-HZSM-5 zeolite. The catalyst of the catalytic cracking unit may contain a mesoporous P2O5-HZSM-5 based spray dried catalyst. In certain embodiments, a weight percent ratio of catalyst to the light cut stream in the catalytic cracking unit ranges from 10:1 to 50:1.

Embodiments of a method for production of light olefins and aromatics from a gas condensate feedstock includes the step of supplying a hydrogen stream and a gas condensate feedstock containing ethane, propane, butane, light naphtha, and nitrogen- and sulfur-containing impurities to a hydrotreating unit for substantial removal of the nitrogen- and sulfur-containing impurities from the gas condensate feedstock to produce a purified feedstock. In certain embodiments, the gas condensate feedstock is substantially all gas condensate with an API gravity ranging from 50 to 70. The method further includes the step of supplying the purified feedstock to a splitter and separating a light cut stream, a medium cut stream, and a heavy cut stream from the purified feedstock, the light cut stream containing a first plurality of hydrocarbons with a boiling point less than 30° C., the medium cut stream containing a second plurality of hydrocarbons with a boiling point ranging from 30° C. to 300° C., the heavy cut stream containing a third plurality of hydrocarbons with a boiling point greater than 300° C. The method further includes the step of supplying the light cut stream to a steam cracker to produce a first olefin product stream containing ethylene, propylene, and aromatics; supplying the medium cut stream to a catalytic cracking unit to produce a cracked product stream containing ethane, propane, and aromatics; supplying a first portion of the cracked product stream to the steam cracker to produce the first olefin product stream containing ethylene, propylene, and aromatics, and supplying a second portion of the cracked product stream to a gas concentration unit for further processing to produce a second olefin product stream containing the propylene and the aromatics. In certain embodiments, a catalyst of the catalytic cracking unit may contain an FCC type based catalyst or USY based catalyst and an FCC additive or Mesoporous P2O5-HZSM-5 based catalyst. In certain embodiments, the FCC catalyst may contain an amorphous, porous solid acid matrix and a USY based zeolite, and the FCC additive may contain a Zeolite Socony Mobil (ZSM)-5 and Mesoporous P2O5-HZSM-5 zeolite. A catalyst of the catalytic cracking unit may contain a mesoporous P2O5-Zeolite Socony Mobil-5-based spray dried catalyst. In certain embodiments, a weight percent ratio of catalyst to the light cut stream in the catalytic cracking unit may range from 10:1 to 50:1. The first olefin product stream may contain about two to ten times more of ethylene and propylene by weight percent as compared to ethylene and propylene content in the cracked product stream. The first olefin product stream may contain about one to five times more of ethylene by weight percent as compared to ethylene content in the cracked product stream. The first olefin product stream may contain about 50 wt. % to about 80 wt. % more of propylene as compared to propylene content in the cracked product stream.

In certain embodiments, the method can further include an optional step of supplying the heavy cut stream from the splitter to a catalytic reformer unit to produce an aromatics-enriched stream and a light hydrocarbon stream; combining the light hydrocarbon stream with the medium cut stream from the splitter before processing in the catalytic cracking unit; and supplying the aromatics-enriched stream to an aromatic recovery complex for production of benzene, toluene, and xylene. In certain embodiments, the method can further include an optional step of recycling a first portion of the aromatics-enriched stream to the hydrotreating unit and supplying a second portion of the aromatics-enriched stream to an aromatic recovery complex for production of benzene, toluene, and xylene. In certain embodiments, the method can further include an optional step of supplying a first portion of the aromatics-enriched stream as a fuel to a regenerator of the catalytic cracking unit and supplying a second portion of the aromatics-enriched stream to an aromatic recovery complex for production of benzene, toluene, and xylene.

In certain embodiments, the method can further include an optional step of supplying the heavy cut stream from the splitter to a catalytic reformer unit to produce an aromatics-enriched stream, a light hydrocarbon stream, and a remaining heavy feed stream; combining the light hydrocarbon stream with the medium cut stream from the splitter before processing in the catalytic cracking unit; supplying the aromatics-enriched stream to an aromatic recovery complex for production of benzene, toluene, and xylene; and supplying the remaining heavy feed stream to the hydrotreater.

In certain embodiments, the method can further include an optional step of supplying the heavy cut stream from the splitter to a catalytic reformer unit to produce an aromatics-enriched stream, a light hydrocarbon stream, and a remaining heavy feed stream; combining the light hydrocarbon stream with the medium cut stream from the splitter before processing in the catalytic cracking unit; supplying the aromatics-enriched stream to an aromatic recovery complex for production of benzene, toluene, and xylene; and supplying the remaining heavy feed stream to a regenerator of the catalytic cracking unit.

Embodiments of an olefin and aromatics production system using a gas condensate feedstock includes a hydrotreating unit configured for receiving a hydrogen stream and a gas condensate feedstock containing ethane, propane, butane, light naphtha, and nitrogen and sulfur containing impurities, removing the nitrogen- and sulfur-containing impurities from the gas condensate feedstock, and producing a purified feedstock. The system further includes a splitter in fluid communication with the hydrotreating unit and configured for receiving the purified feedstock and separating the purified feedstock into a light cut stream and a heavy cut stream, the light cut stream containing a first plurality of hydrocarbons with a boiling point less than about 300° C. and the heavy cut stream containing a second plurality of hydrocarbons with a boiling point greater than 300° C. The system further includes a catalytic cracking unit in fluid communication with the splitter and configured for receiving the light cut stream and producing a cracked product stream containing a portion of the ethane, the propane, and the aromatics; a steam cracker in fluid communication with the catalytic cracking unit and configured for receiving a first portion of the cracked product stream and producing a first olefin product stream containing ethylene, propylene, and aromatics; and a gas concentration unit in fluid communication with the catalytic cracking unit and configured for receiving a second portion of the cracked product stream and producing a second olefin product stream containing the propylene and the aromatics.

The system can further include an optional catalytic reformer unit in fluid communication with the splitter and configured for receiving the heavy cut stream and producing an aromatics-enriched stream and a light hydrocarbon stream; and an aromatic recovery complex in fluid communication with catalytic reformer unit and configured for receiving the aromatics-enriched stream and producing benzene, toluene, and xylene. The light hydrocarbon stream can be combined with the light cut stream from the splitter prior to the catalytic cracking unit.

Embodiments of an olefin and aromatics production system using a gas condensate feedstock includes a hydrotreating unit configured for receiving a hydrogen stream and a gas condensate feedstock containing ethane, propane, butane, light naphtha, and nitrogen and sulfur containing impurities, removing the nitrogen- and sulfur-containing impurities from the gas condensate feedstock, and producing a purified feedstock. The system further includes a splitter in fluid communication with the hydrotreating unit and configured for receiving the purified feedstock and separating the purified feedstock into a light cut stream, a medium cut stream, and a heavy cut stream, the light cut stream containing a first plurality of hydrocarbons with a boiling point less than about 30° C., the medium cut stream containing a second plurality of hydrocarbons with a boiling point ranging from 30° C. to 300° C., the heavy cut stream containing a third plurality of hydrocarbons with a boiling point greater than 300° C. The system further includes a catalytic cracking unit in fluid communication with the splitter and configured for receiving the medium cut stream and producing a cracked product stream containing a portion of the ethane, the propane, and the aromatics; a steam cracker in fluid communication with the splitter and the catalytic cracking unit and configured for receiving the light cut stream and a first portion of the cracked product stream, respectively, and producing a first olefin product stream containing ethylene, propylene, and aromatics; and a gas concentration unit in fluid communication with the catalytic cracking unit and configured for receiving a second portion of the cracked product stream and producing a second olefin product stream containing propylene and aromatics. The system can further include an optional catalytic reformer unit in fluid communication with the splitter and configured for receiving the heavy cut stream and producing an aromatics-enriched stream and a light hydrocarbon stream; and an optional aromatic recovery complex in fluid communication with the catalytic reformer unit and configured for receiving the aromatics-enriched stream and producing benzene, toluene, and xylene. The light hydrocarbon stream may be combined with the medium cut stream from the splitter prior to the catalytic cracking unit.

Certain embodiments of the systems can include an optional catalytic reformer unit in fluid communication with the splitter and configured for receiving the heavy cut stream and producing an aromatics-enriched stream, a light hydrocarbon stream, and a remaining heavy feed stream; and an aromatic recovery complex in fluid communication with catalytic reformer unit and configured for receiving the aromatics-enriched stream and producing benzene, toluene, and xylene. The light hydrocarbon stream may be combined with the medium cut stream from the splitter before processing in the catalytic cracking unit. The remaining heavy feed stream may be recycled to the hydrotreater. In certain embodiments, the remaining heavy feed stream may contain unconverted feed. The remaining heavy feed stream may be supplied to a regenerator of the catalytic cracking unit as fuel.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A method for production of light olefins and aromatics from a gas condensate feedstock, the method comprising:
supplying a hydrogen stream and a gas condensate feedstock containing ethane, propane, butane, light naphtha, and nitrogen and sulfur containing impurities to a hydrotreating unit for substantial removal of the nitrogen and sulfur containing impurities from the gas condensate feedstock to produce a purified feedstock;
supplying the purified feedstock to a splitter and separating a light cut stream and a heavy cut stream from the purified feedstock, the light cut stream containing a first plurality of hydrocarbons with a boiling point less than about 300° C. and the heavy cut stream containing a second plurality of hydrocarbons with a boiling point greater than 300° C.;
supplying the light cut stream to a catalytic cracking unit to produce a cracked product stream containing a portion of the ethane, the propane, and the aromatics;
supplying a first portion of the cracked product stream to a steam cracker to produce a first olefin product stream containing ethylene, propylene, and the aromatics, and supplying a second portion of the cracked product stream to a gas concentration unit for further processing to produce a second olefin product stream containing the propylene and the aromatics;
supplying the heavy cut stream from the splitter to a catalytic reformer unit to produce an aromatics-enriched stream and a light hydrocarbon stream;
combining the light hydrocarbon stream with the light cut stream from the splitter before processing in the catalytic cracking unit; and
supplying the aromatics-enriched stream to an aromatic recovery complex for production of benzene, toluene, and xylene.

2. The method of claim 1, wherein the gas condensate feedstock is substantially all gas condensate with an API gravity ranging from 45 to 70.

3. The method of claim 1, further comprising:
recovering a remaining heavy feed stream from the catalytic reformer unit; and
recycling the remaining heavy feed stream to the hydrotreating unit.

4. The method of claim 1, further comprising:
recovering a remaining heavy feed stream from the catalytic reformer unit; and
supplying the remaining heavy feed stream as a fuel to a regenerator of the catalytic cracking unit.

5. The method of claim 1, wherein a catalyst of the catalytic cracking unit contains an FCC type based catalyst or a USY based catalyst and an FCC additive or a Mesoporous P2O5-HZSM-5 based catalyst, the FCC type based catalyst contains an amorphous, porous solid acid matrix and a USY based zeolite, and the FCC additive contains a Zeolite Socony Mobil (ZSM)-5 and a Mesoporous P₂O₅-HZSM-5 zeolite.

6. The method of claim 1, wherein a catalyst of the catalytic cracking unit contains a mesoporous P₂O₅-HZSM-5 based spray dried catalyst.

7. The method of claim 1, wherein a weight percent ratio of catalyst to the light cut stream in the catalytic cracking unit ranges from 10:1 to 50:1.

8. The method of claim 1, wherein the first olefin product stream contains about two to ten times weight percent more of ethylene and propylene by weight percent as compared to the cracked product stream.

9. An olefin and aromatics production system using a gas condensate feedstock, the system comprising:
a hydrotreating unit configured for receiving a hydrogen stream and a gas condensate feedstock containing ethane, propane, butane, light naphtha, and nitrogen and sulfur containing impurities, removing the nitrogen- and sulfur-containing impurities from the gas condensate feedstock, and producing a purified feedstock;
a splitter in fluid communication with the hydrotreating unit and configured for receiving the purified feedstock and separating the purified feedstock into a light cut stream and a heavy cut stream, the light cut stream containing a first plurality of hydrocarbons with a boiling point less than about 300° C. and the heavy cut stream containing a second plurality of hydrocarbons with a boiling point greater than 300° C.;
a catalytic cracking unit in fluid communication with the splitter and configured for receiving the light cut stream and producing a cracked product stream containing a portion of the ethane, the propane, and the aromatics;
a steam cracker in fluid communication with the catalytic cracking unit and configured for receiving a first portion of the cracked product stream and producing a first olefin product stream containing ethylene, propylene, and the aromatics;
a gas concentration unit in fluid communication with the catalytic cracking unit and configured for receiving a second portion of the cracked product stream and producing a second olefin product stream containing the propylene and the aromatics;
a catalytic reformer unit in fluid communication with the splitter and configured for receiving the heavy cut stream and producing an aromatics-enriched stream and a light hydrocarbon stream, the light hydrocarbon stream to combine with the light cut stream from the splitter prior to the catalytic cracking unit; and
an aromatic recovery complex in fluid communication with the catalytic reformer unit and configured for receiving the aromatics-enriched stream and producing benzene, toluene, and xylene.

10. The system of claim 9, wherein the gas condensate feedstock is substantially all gas condensate with an API gravity ranging from 45 to 70.

11. The system of claim 9, wherein the catalytic reformer unit is configured for receiving the heavy cut stream and producing the aromatics-enriched stream, the light hydrocarbon stream, and a remaining heavy feed stream.

12. The system of claim 11, wherein the remaining heavy feed stream is recycled to the hydrotreating unit.

13. The system of claim 11, wherein the remaining heavy feed stream is supplied as a fuel to a regenerator of the catalytic cracking unit.

14. The system of claim 9, wherein a weight percent ratio of catalyst to the light cut stream in the catalytic cracking unit ranges from 10:1 to 50:1.

15. The system of claim 9, wherein the first olefin product stream contains about two to ten times weight percent more of ethylene and propylene by weight percent as compared to the cracked product stream.
